(19) United States
(12) Patent Application Publication
(10) Pub. No.: US 2014/0309221 A1
(43) Pub. Date: Oct. 16, 2014

(54) METHOD OF INHIBITING HAMARTOMA TUMOR CELLS

(71) Applicants: Jean J. Zhao, Brookline, MA (US); Qi Wang, Boston, MA (US)

(72) Inventors: Jean J. Zhao, Brookline, MA (US); Qi Wang, Boston, MA (US)

(73) Assignee: DANA-FARBER CANCER INSTITUTE, INC., Boston, MA (US)

(21) Appl. No.: 13/984,422

(22) PCT Filed: Feb. 9, 2012
(86) PCT No.: PCT/US12/24440

§ 371 Date: Oct. 18, 2013

Related U.S. Application Data
(60) Provisional application No. 61/441,896, filed on Feb. 11, 2011.

(51) Int. Cl. C07D 401/04

(52) U.S. Cl. CPC C07D 401/04
USPC 514/232.2

(57) ABSTRACT

Dimorpholinopyrimidines are useful for inhibiting growth or proliferation of hamartoma tumor cells. Because the Dimorpholinopyrimidines inhibit the growth and proliferation of hamartoma tumor cells they are also useful in treating PTEN hamartoma tumor syndromes. The therapeutic and prophylactic treatments provided by this invention are practiced by administering to a patient in need thereof an amount of a compound of dimorpholinopyrimidine derivative that is effective to inhibit growth or proliferation of the hamartoma tumor cells.

### RELATED APPLICATIONS

This application claims the benefit of the filing date under 35 U.S.C. §119(e) of the Provisional U.S. Patent Application Ser. No. 61/441,896, filed Feb. 11, 2011, which is hereby incorporated by reference in its entirety.

### FEDERALLY SPONSORED RESEARCH OR DEVELOPMENT

The present invention described herein was supported at least in part by contract number R01 CA134502 awarded by the U.S. National Institutes of Health (NIH) and the National Cancer Institute (NCI). The U.S. government may retain certain rights in the invention.

### BACKGROUND OF THE INVENTION

The PTEN hamartoma tumor syndromes (PHTS) are a collection of rare and disparate disorders associated with germline mutations in the tumor suppressor gene PTEN (phosphatase and tensin homolog, deleted on chromosome 10). These syndromes are characterized by cellular overgrowth leading to benign hamartomas in virtually any organ. PTEN encodes a dual phosphatase protein that negatively regulates the PI3K/Akt/mTOR pathway. Somatic loss of PTEN function through mutation, deletion, or methylation has been described in various sporadic human cancers, including those of the brain, breast, prostate, colon, lung, and endometrium, and is thus under investigation by cancer researchers. Blumenthal, G. M. and Dennis, P. A., Eur. J. Hum. Gen. 16, 1289-1300 (2008).

Hamartomas are a histologically distinct subtype of benign tumors in which cells maintain normal differentiation but are disorganized with respect to architecture. Cowden syndrome (CS) is the prototypic syndrome, characterized by mucocutaneous lesions, benign hamartomas, macrocephaly, and increased predisposition to breast, thyroid, and endometrial carcinoma. Lhermitte-Duclos (LD), a variant of CS, is characterized by dysplastic gangliocytomas of the cerebellum, which can lead to hydrocephalus, ataxia, and seizures. After the discovery of the PTEN gene and the fact that CS is caused by germline mutations of PTEN, it became apparent that CS is allelic to other seemingly unrelated clinical syndromes. Bannayan-Riley-Ruvalcaba syndrome (BRRS), characterized by the developmental delay, macrocephaly, lipomas, hemangiomas, and speckled penis in males, is associated with PTEN mutations in approximately 60% of cases. Proteus syndrome has also been associated with germline PTEN mutations, although this is controversial. The clinical management of PHTS patients has historically focused on genetic counseling and screening. Patients with PHTS, particularly those with CS, should undergo early and frequent surveillance for susceptible malignancies. No medical therapies currently exist for PHTS patients.

The PTEN gene (also known as MMAC1 or TEP1) spans nine exons and is located on chromosome 10q22-23. The gene encodes a 403 amino-acid protein, which acts as a dual specificity phosphatase that dephosphorylates lipids and proteins. PTEN exerts its lipid phosphatase activity by dephosphorylating the 30-phosphoinositide products of PI3K, causing conversion of phosphatidylinositol (3,4,5) trisphosphate to phosphatidylinositol (4,5) bisphosphate and conversion of phosphatidylinositol (3,4) bisphosphate to phosphatidylinositol (4) phosphate. Reduction of 30-phosphoinositides decreases activity of kinases downstream of PI3K such as phosphoinositide-dependent kinase 1, Akt, and mTOR, and is responsible for its tumor suppressor activity. Because of negative regulation of the Aid pathway, PTEN indirectly decreases phosphorylation of other substrates downstream of Aid such as p27, p21, GSK-3, Bad, ASK-1, as well as members of the forkhead transcription factor family (eg, AFX, FKHR, FKHRL1). Thus, a loss or reduction in PTEN activity leads to increased phosphorylation of many key cellular proteins, which in turn can affect processes such as cell cycle progression, metabolism, migration, apoptosis, transcription, and translation.

G. M. Blumenthal and P. A. Dennis hypothesize that many different types of therapies may be useful in treating PHTS including inhibitors of the PI3K/Akt/mTOR pathway, rapamycin, highly specific mTOR inhibitors, agents that bind FKBP12, proteosome inhibitors, including bortezomib, and PINK1 agonists. However, Blumenthal and Dennis do not teach any specific therapy for PHTS and indicate that there will likely be significant hurdles in developing effective therapeutics.

It is an object of the present invention to provide therapeutic agents that inhibit the growth or proliferation of hamartoma tumor cells. It is another object of the invention to treat PTEN hamartoma tumor syndromes.

### SUMMARY OF THE INVENTION

The present application is directed to a method for inhibiting growth or proliferation of hamartoma tumor cells comprising administering to a patient in need thereof in an amount that is effective to inhibit growth or proliferation of the hamartoma tumor cells a compound of the formula

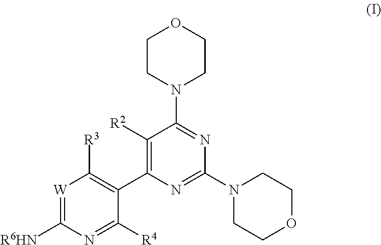

wherein R² is hydrogen or halogen; R³ is hydrogen, cyano, nitro, halogen, hydroxyl, amino, or trifluoromethyl; R⁴ is hydrogen or halogen; R⁶ is hydrogen, methyl, or ethyl; and W is CR_{w} or N, wherein R_{w} is hydrogen, cyano, halogen, methyl, trifluoromethyl, or sulfonamido; or a pharmaceutically acceptable salt thereof.

The present invention is also directed to a method for treating a PTEN hamartoma tumor syndrome comprising administering to a patient in need thereof in an amount that is effective to inhibit growth or proliferation of the hamartoma tumor cells a compound of formula (I) or a pharmaceutically acceptable salt thereof.

### BRIEF DESCRIPTION OF THE DRAWINGS

FIG. 1a is a series of photographs illustrating that ablation of p110-alpha and p110-beta isoforms of PI3K blocked development of PHTS in a mouse model of PHTS. (i) K14-cre Pten f/f (n=28), (ii) K14-cre Pten f/f; p110a f/f (n=16), (iii) K14-cre Pten f/f; p110b f/f (n=11) and (iv) k14-cre-Pten f/f; p110a f/f; p110b f/f (n=15) mice. Ablation of only one of the isoforms shows only partial inhibition of PHTS.

FIG. 1b is a Kaplan-Meier plot of the onset of PHTS in the (i) K14-cre Pten f/f (n=28), (ii) K14-cre Pten f/f; p110a f/f (n=16), (iii) K14-cre Pten f/f; p110b f/f (n=11) and (iv) k14-cre-Pten f/f; p110a f/f; p110b f/f (n=15) mice. Median PHTS onset:

| | |
|---|---|
| K14-cre Pten f/f | 62 days |
| K14-cre Ptenf/f; p110a f/f | 134 days |
| f K14-cre Ptenf/f; p110b f/f | 121 days |

FIG. 2a is a series of photographs demonstrating that mice treated with 4-(trifluoromethyl)-5-(2,6-dimorpholinopyrimidin-4-yl)pyridin-2-amine (COMPOUND A) remained free of PHTS symptoms (ii); while mice treated with vehicle alone developed characteristic PHTS lesions on their face and limbs (i).

FIG. 2b represents a Kaplan-Meier curve of PHTS free survival in K14-cre-Pten f/f mice (n=12) maintained with 4-(trifluoromethyl)-5-(2,6-dimorpholinopyrimidin-4-yl)pyridin-2-amine (COMPOUND A) as described above (ii); or treated with vehicle only (i).

FIG. 3 represents a series of photographs showing the heads and front left paws of two K14-cre-Pten f/f mice treated daily with 45 mg/ml 4-(trifluoromethyl)-5-(2,6-dimorpholinopyrimidin-4-yl)pyridin-2-amine (COMPOUND A). FIG. 3 illustrates the effects of drug treatment over time on a mouse with fully-developed PHTS.

### DETAILED DESCRIPTION OF THE INVENTION

The current invention relates to the discovery that a select group of substituted 2,6-dimorpholinopyrimidines, as set forth in formula (I), are useful for inhibiting growth or proliferation of hamartoma tumor cells. Because the 2,6-dimorpholinopyrimidines of formula (I) inhibit the growth and proliferation of hamartoma tumor cells they are also useful in treating PTEN hamartoma tumor syndromes. The therapeutic and prophylactic treatments provided by this invention are practiced by administering to a patient in need thereof an amount of a compound of formula (I) that is effective to inhibit growth or proliferation of the hamartoma tumor cells.

The term “halogen” refers to fluorine, chlorine, bromine, and iodine.

As used herein, the term “inhibit”, “inhibiting”, or “inhibit the growth or proliferation” of the hamartoma tumor cell refers to slowing, interrupting, arresting or stopping the growth of the hamartoma tumor cell, and does not necessarily indicate a total elimination of the hamartoma tumor cell growth. The terms “inhibit” and “inhibiting”, or the like, denote quantitative differences between two states, refer to at least statistically significant differences between the two states. For example, “an amount effective to inhibit growth of hamartoma tumor cells” means that the rate of growth of the cells will be at least statistically significantly different from the untreated cells. Such terms are applied herein to, for example, rates of cell proliferation

“Treating”, “treat”, or “treatment” within the context of the instant invention, means an alleviation of symptoms associated with a disorder or disease, or halt of further progression or worsening of symptoms. For example, within the context of this invention, successful treatment may include an alleviation of symptoms related to a hamartoma tumor or a halting in the progression of a disease such as PHTS. In certain circumstances, treatment may also include the identification of asymptomatic patients who are at risk of developing hamartoma tumors or PHTS.

“Hamartomas” or “hamartoma tumors” refer to a histologically distinct subtype of benign tumors in which cells maintain normal differentiation but are disorganized with respect to architecture.

“PTEN hamartoma tumor syndromes” or “PHTS” refer to a spectrum of syndromes with variable clinical manifestations characterized by aberrant growth and associated with germline PTEN mutations. Cowden syndrome (CS), Lhermitt-Duclos syndrome (LD), Bannayan-Riley-Ruvalcaba syndrome, and Proteus syndrome are all examples of PHTS.

As used herein, the term “pharmaceutically acceptable salts” refers to the nontoxic acid or alkaline earth metal salts of the pyrimidine compounds of the invention. These salts can be prepared in situ during the final isolation and purification of the pyrimidine compounds, or by separately reacting the base or acid functions with a suitable organic or inorganic acid or base, respectively. Representative salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemi-sulfate, heptanoate, hexanoate, fumarate, hydrochloride, hydrobromide, hydroiodide, 2-hydroxyethanesulfonate, lactate, maleate, methanesulfonate, nicotinate, 2-naphth-alenesulfonate, oxalate, pamoate, pectinate, persulfate, 3-phenylproionate, picrate, pivalate, propionate, succinate, sulfate, tartrate, thiocyanate, p-toluenesulfonate, and undecanoate. Also, the basic nitrogen-containing groups can be quaternized with such agents as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl, and stearyl chlorides, bromides and iodides, aralkyl halides like benzyl and phenethyl bromides, and others. Water or oil-soluble or dispersible products are thereby obtained.

Examples of acids that may be employed to form pharmaceutically acceptable acid addition salts include such inorganic acids as hydrochloric acid, hydroboric acid, nitric acid, sulfuric acid and phosphoric acid and such organic acids as formic acid, acetic acid, trifluoroacetic acid, fumaric acid, tartaric acid, oxalic acid, maleic acid, methanesulfonic acid, succinic acid, malic acid, methanesulfonic acid, benzenesulfonic acid, and p-toluenesulfonic acid, citric acid, and acidic amino acids such as aspartic acid and glutamic acid.

Basic addition salts can be prepared in situ during the final isolation and purification of the pyrimidine compounds, or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine, pyridine, picoline, triethanolamine and the like, and basic amino acids such as arginine, lysine and ornithine.

The compounds of formula (I) may be used alone or in compositions together with a pharmaceutically acceptable carrier or excipient. Pharmaceutical compositions of the present invention comprise a therapeutically effective amount of a compound of formula (I) formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, incorporated herein by reference.

The compounds of formula (I) may be administered to humans and other animals orally, parenterally, sublingually, by aerosolization or inhalation spray, rectally, intracisternally, intravaginally, intraperitoneally, bucally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules, suppositories, lyophilized powders, transdermal patches or other forms known in the art.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-propanediol or 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compositions of the invention may also be formulated for delivery as a liquid aerosol or inhalable dry powder. Liquid aerosol formulations may be nebulized predominantly into particle sizes that can be delivered to the terminal and respiratory bronchioles.

Aerosolized formulations of the invention may be delivered using an aerosol forming device, such as a jet, vibrating porous plate or ultrasonic nebulizer, preferably selected to allow the formation of an aerosol particles having with a mass medium average diameter predominantly between 1 to 5 μm. Further, the formulation preferably has balanced osmolarity ionic strength and chloride concentration, and the smallest aerosolizable volume able to deliver effective dose of the compounds of the invention to the site of the infection. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, ultrasonic nebulizers and energized dry powder inhalers, that are able to nebulize the formulation of the invention into aerosol particle size predominantly in the size range from 1-5 μm. Predominantly in this application means that at least 70% but preferably more than 90% of all generated aerosol particles are within 1-5 μm range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AERONEB and AERODOSE vibrating porous plate nebulizers (AeroGen, Inc., Sunnyvale, Calif.), SIDESTREAM nebulizers (Medic-Aid Ltd., West Sussex, England), PARI LC and PARI LC STAR jet nebulizers (Pari Respiratory Equipment, Inc., Richmond, Va.), and AEROSONIC (DeVilbiss Medizinische Produkte (Deutschland) GmbH, Heiden, Germany) and ULTRAAIRE (Omron Healthcare, Inc., Vernon Hills, Ill.) ultrasonic nebulizers.

Compounds of the invention may also be formulated for use as topical powders and sprays that can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott (ed.), “Methods in Cell Biology,” Volume XIV, Academic Press, New York, 1976, p. 33 et seq.

Effective amounts of the compounds of the invention generally include any amount sufficient to detectably inhibit the growth or proliferation of hamartoma tumor cells, or by detecting an inhibition or alleviation of symptoms of PHTS. The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

According to the methods of treatment of the present invention, hamartoma tumor growth is reduced or prevented in a patient such as a human or lower mammal by administering to the patient an amount of a compound of formula (I), in such amounts and for such time as is necessary to achieve the desired result. An “amount that is effective to inhibit growth or proliferation of the hamartoma tumor cells” of a compound of formula (I) refers to a sufficient amount of the compound to treat hamartoma tumor growth, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

For purposes of the present invention, a therapeutically effective dose will generally be a total daily dose administered to a host in single or divided doses may be in amounts, for example, of from 0.001 to 1000 mg/kg body weight daily and more preferred from 1.0 to 30 mg/kg body weight daily. Dosage unit compositions may contain such amounts of submultiples thereof to make up the daily dose. In general, treatment regimens according to the present invention comprise administration to a patient in need of such treatment from about 10 mg to about 2000 mg of the compound(s) of this invention per day in single or multiple doses.

Alternate embodiments of the compounds of formula (I) are given below:

1) Compounds where R² is:
a. hydrogen;
b. hydrogen or fluorine; or
c. hydrogen, fluorine, or chlorine;

2) Compounds where R³ is:
a. trifluoromethyl;
b. hydrogen or trifluoromethyl;
c. hydrogen, halogen, or trifluoromethyl;

3) Compounds where R⁴ is:
a. hydrogen; or
b. hydrogen, fluorine, or chlorine;

4) Compounds where R⁶ is hydrogen;

5) Compounds where W is:
a. CH; or
b. N.

It is understood that additional embodiments of the compounds of formula (I) can be selected by requiring one or more of the embodiments (1) through (5) above of the compounds of formula (I). For example, further alternate embodiments can be obtained by combining (1)(a) and (2)(a); (1)(a) and (2)(b); (1)(b) and (2)(a); (1)(b) and (2)(b); (1)(c) and (2)(a); (1)(c) and (2b); (1)(c) and (2)(c); (1)(b) and (2)(c); (1)(a) and (2)(c); (1)(a), (2)(a), and (3)(a); (1)(b), (2)(a), and (3)(a); (1)(a), (2)(b), and (3)(a); (1)(a), (2)(a), and (3)(b); (1)(b), (2)(b), and (3)(a); (1)(a), (2)(b), and (3)(b); (1)(a), (2)(a), (3)(a), and (4); (1)(b), (2)(a), (3)(a), and (4); (1)(a), (2)(a), (3)(b), and (4); (1)(b), (2)(b), (3)(a), and (4); (1)(b), (2)(a), (3)(b), and (4); (1)(b), (2)(b), (3)(b), and (4); (1)(c), (2)(a), (3)(a), and (4); (1)(c), (2)(b), (3)(a), and (4); (1)(c), (2)(b), (3)(b), and (4); (1)(c), (2)(c), (3)(a), and (4); (1)(c), (2)(c), (3)(b), and (4); (1)(a), (2)(a), (3)(a), (4), and (5)(a); (1)(b), (2)(a), (3)(a), (4), and (5)(a); (1)(a), (2)(b), (3)(a), (4), and (5)(a); (1)(a), (2)(a), (3)(b), (4), and (5)(a); (1)(b), (2)(b), (3)(a), (4), and (5)(a); (1)(b), (2)(b), (3)(a), (4), and (5)(a); (1)(c), (2)(a), (3)(a), (4), and (5)(a); (1)(a), (2)(c), (3)(a), (4), and (5)(a); (1)(c), (2)(c), (3)(b), (4), and (5)(a); (1)(a), (2)(a), (3)(a), (4), and (5)(b); (1)(b), (2)(a), (3)(a), (4), and (5)(b); (1)(a), (2)(b), (3)(a), (4), and (5)(b); (1)(a), (2)(a), (3)(b), (4), and (5)(b); (1)(c), (2)(a), (3)(a), (4), and (5)(b); (1)(c), (2)(a), (3)(a), (4), and (5)(b); (1)(c), (2)(b), (3)(a), (4), and (5)(b); (1)(b), (2)(c), (3)(a), (4), and (5)(b); (1)(b), (2)(b), (3)(b), (4), and (5)(b); (1)(c), (2)(c), (3)(b), (4), and (5)(b); (1)(a) and (4); (1)(b) and (4); (2)(a) and (4); (3)(a) and (4); (2)(a), (3)(a), and (4); (1)(a) and (5)(a); (1)(b), (4) and (5); and the like.

The present invention will be understood more readily by reference to the following examples, which are provided by way of illustration and are not intended to be limiting of the present invention.

The compounds of formula (I) were synthesized using the methods described in U.S. Patent Application Publication No. US 2010/0249126 A1, published Sep. 30, 2010, which is hereby incorporated by reference as if fully set forth. Select examples are also disclosed herein as set forth in the Schemes and Examples below.

The compounds and/or intermediates were characterized by high performance liquid chromatography (HPLC) using a Waters Millenium chromatography system with a 2695 Separation Module (Milford, Mass.). The analytical columns were Alltima C-18 reversed phase, 4.6×50 mm, flow 2.5 mL/min, from Alltech (Deerfield, Ill.). A gradient elution was used, typically starting with 5% acetonitrile/95% water and progressing to 100% acetonitrile over a period of 40 minutes. All solvents contained 0.1% trifluoroacetic acid (TFA). Compounds were detected by ultraviolet light (UV) absorption at either 220 or 254 nm. HPLC solvents were from Burdick and Jackson (Muskegan, Mich.), or Fisher Scientific (Pittsburgh, Pa.). In some instances, purity was assessed by thin layer chromatography (TLC) using glass or plastic backed silica gel plates, such as, for example, Baker-Flex Silica Gel 1B2-F flexible sheets. TLC results were readily detected visually under ultraviolet light, or by employing well known iodine vapor and other various staining techniques.

Mass spectrometric analysis was performed on one of two LCMS instruments: a Waters System (Alliance HT HPLC and a Micromass ZQ mass spectrometer; Column: Eclipse XDB-C18, 2.1×50 mm; solvent system: 5-95% (or 35-95%, or 65-95% or 95-95%) acetonitrile in water with 0.05% TFA; flow rate 0.8 mL/min; molecular weight range 200-1500; cone Voltage 20 V; column temperature 40° C.) or a Hewlett Packard System (Series 1100 HPLC; Column: Eclipse XDB-C18, 2.1×50 mm; solvent system: 1-95% acetonitrile in water with 0.05% TFA; flow rate 0.8 mL/min; molecular weight range 150-850; cone Voltage 50 V; column temperature 30° C.). All masses were reported as those of the protonated parent ions.

GCMS analysis is performed on a Hewlett Packard instrument (HP6890 Series gas chromatograph with a Mass Selective Detector 5973; injector volume: 1 μL; initial column temperature: 50° C.; final column temperature: 250° C.; ramp time: 20 minutes; gas flow rate: 1 mL/min; column: 5% phenyl methyl siloxane, Model No. HP 190915-443, dimensions: 30.0 m×25 m×0.25 m).

Nuclear magnetic resonance (NMR) analysis was performed on some of the compounds with a Varian 300 MHz NMR (Palo Alto, Calif.). The spectral reference was either TMS or the known chemical shift of the solvent. Some compound samples were run at elevated temperatures (e.g., 75° C.) to promote increased sample solubility.

The purity of some of the invention compounds is assessed by elemental analysis (Desert Analytics, Tucson, Ariz.).

Melting points are determined on a Laboratory Devices MeI-Temp apparatus (Holliston, Mass.).

Preparative separations were carried out using a Flash 40 chromatography system and KP-Sil, 60A (Biotage, Charlottesville, Va.), or by flash column chromatography using silica gel (230-400 mesh) packing material, or by HPLC using a Waters 2767 Sample Manager, C-18 reversed phase column, 30×50 mm, flow 75 mL/min. Typical solvents employed for the Flash 40 Biotage system and flash column chromatography were dichloromethane, methanol, ethyl acetate, hexane, acetone, aqueous ammonia (or ammonium hydroxide), and triethyl amine. Typical solvents employed for the reverse phase HPLC were varying concentrations of acetonitrile and water with 0.1% trifluoroacetic acid.

It should be understood that the organic compounds according to the invention may exhibit the phenomenon of tautomerism. As the chemical structures within this specification can only represent one of the possible tautomeric forms, it should be understood that the invention encompasses any tautomeric form of the drawn structure.

It is understood that the invention is not limited to the embodiments set forth herein for illustration, but embraces all such forms thereof as come within the scope of the above disclosure.

Abbreviations

ACN Acetonitrile

BINAP 2,2′-bis(diphenylphosphino)-1,1′-binapthyl

DIEA diisopropylethylamine

DME 1,2-dimethoxyethane

DMF N,N-dimethylformamide

DPPF 1,1′-bis(diphenylphosphino)ferrocene

EtOAc ethyl acetate

EtOH ethanol

MCPBA meta-chloroperoxybenzoic acid

NBS N-bromosuccinimide

NMP N-methyl-2-pyrrolidone

RT room temperature

THF tetrahydrofuran

General Methods for Synthesizing Formula (I) Compounds

Methods for preparing compounds of formula (I) are provided. The methods include: reacting a 4-halo-2,6-dimorpholinopyrimidine with a substituted pyridinyl or pyrimidinyl group containing a reactive boronic ester substituent, in the presence of a palladium catalyst. In one embodiment, the substituted pyridinyl or pyrimidinyl group containing a reactive boronic ester substituent has an —NH₂ group positioned para to the boronic ester. In another embodiment, the substituted pyridinyl or pyrimidinyl group containing a reactive boronic ester substituent has an —NH₂ group positioned para to the boronic ester and another non-hydrogen substituent positioned ortho to the boronic ester. In certain embodiments, the non-hydrogen substituent is —CF₃, —CN, —NH₂, halogen, hydroxyl or nitro.

In another embodiment, the pyridinyl boronic ester is 4-(trifluoromethyl)-5-(4,4,5,5-tetramethyl-1,3,2-dioxaborolan-2-yl)pyridin-2-amine. In another embodiment, the palladium catalyst is Pd(dppf)Cl₂ dichloromethane adduct.

In another embodiment, the 4-chloro-2,6-dimorpholinopyrimidine group is prepared by reacting 4,6-dichloro-2-morpholinopyrimidine with morpholine. In another embodiment, the 4,6-dichloro-2-morpholinopyrimidine group is prepared by reacting 2-morpholinopyrimidine-4,6-diol with POCl₃. In another embodiment, the 2-morpholinopyrimidine-4,6-diol is prepared by reacting morpholine-4-carboxamidine with diethyl malonate in the presence of a base, such as sodium ethoxide.

In another embodiment, the substituted pyridinyl or pyrimidinyl group containing a reactive boronic ester substituent is prepared by reacting a substituted pyridinyl or pyrimidinyl group containing a bromo substituent with a diboronic ester, such as 4,4,5,5-tetramethyl-2-(4,4,5,5-tetramethyl-1,3,2-dioxaborolan-2-yl)-1,3,2-dioxaborolane. In another embodiment, the substituted pyridinyl or pyrimidinyl group containing a bromo substituent is prepared by reacting the substituted pyridinyl or pyrimidinyl group with N-bromosuccinimide (NBS).

Another embodiment of the present invention provides a method of preparing a 4-chloro-2,6-dimorpholinopyrimidine comprising reacting morpholine with 2,4,6-trichloropyrimidine in a suitable solvent. In a more particular embodiment, the solvent is a polar aprotic solvent. More particular still the solvent is THF. In another more particular embodiment, the 4-chloro-2,6-dimorpholinopyrimidine is added over a period of at least 10 minutes, or at least 20 minutes, or 30 minutes to a solution comprising morpholine. Alternatively, the morpholine is added to a solution comprising 4-chloro-2,6-dimorpholinopyrimidine. More particular still, the solution is cooled below 20° C., or below 10° C., or below 5° C., or below 0° C. More particularly, during or after addition of the 4-chloro-2,6-dimorpholinopyrimidine, the solution is allowed to warm to greater than 20° C., or greater than 25° C., or greater than 30° C. In another embodiment, after the morpholine and 4-chloro-2,6-dimorpholinopyrimidine are combined, the solution is quenched by addition of an aqueous solution. More particularly, at least 10 hours, or at least 20 hours, or at least 30 hours, or at least 40 hours, or at least 50 hours, or about 64 hours after the morpholine and 4-chloro-2,6-dimorpholinopyrimidine are combined, the solution is quenched by addition of an aqueous solution. More particularly, after quenching, the solution is purified by column chromatography. More particular still, the column is silica gel. In another embodiment, the 4-chloro-2,6-dimorpholinopyrimidine is reacted with a 2-aminopyridyl or 2-aminopyrimidyl moiety to form a compound of formula (I).

### Method 1

### Synthesis of 5-(4,4,5,5-tetramethyl-1,3,2-dioxaborolan-2-yl)pyrimidine-2-ylamine

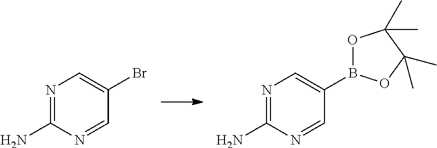 [SMILES: Nc1ncc(Br)cn1 ; CC1(C)OB(c2cnc(N)nc2)OC1(C)C]

To a dry 500-mL flask was added 2-amino-5-bromopyrimidine (10 g, 57.5 mmol), potassium acetate (16.9 g, 172 mmol), 4,4,5,5-tetramethyl-2-(4,4,5,5-tetramethyl-1,3,2-dioxaborolan-2-yl)-1,3,2-dioxaborolane (16.1 g, 63.0 mmol) and dioxane (300 mL). Argon was bubbled through the solution for 15 minutes, at which time dichloro[1,1′-bis(diphenylphosphino)ferrocene]palladium (II) dichloromethane adduct (Pd(dppf)Cl₂ CH₂Cl₂) (2.34 g, 2.87 mmol) was added. The reaction mixture was refluxed in a 115° C. oil bath for 4 hours under argon. After cooling to room temperature, EtOAc (500 mL) was added and the resulting slurry was sonicated and filtered. Additional EtOAc (500 mL) was used to wash the solid. The combined organic extracts were washed with H₂O (2×300 mL), NaCl_{(sat.)} (300 mL), dried over Na₂SO₄, and filtered through a 5 cm pad of silica gel. Additional EtOAc was used to flush product. After the solvent was concentrated, the crude was treated with a mixture of 1:3 dichloromethane and hexane (40 mL), filtered and washed with hexane yielding a light yellow solid (8.5 g, 75%). LCMS (m/z): 140 (MH⁺ of boronic acid, deriving from product hydrolysis on LC). ¹H NMR (CDCl₃): δ 8.58 (s, 2H), 5.74 (s, 2H), 1.32 (s, 12H).

### Method 2

### Synthesis of 2-Aminomethyl-5-bromopyrimidine

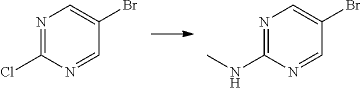

Methylamine (2.0 M in methanol, 40 mL, 80 mmol) was added to 5-bromo-2-chloropyrimidine (5.6 g, 29.0 mmol) in a sealable reaction vessel. After allowing to vent for a few minutes, the vessel was sealed, placed behind a safety shield and heated in a 115° C. oil bath for 48 hours. Upon cooling the volatiles were removed in vacuo. The material was dissolved in CH₂Cl₂ (200 mL) and washed with 1M NaOH (40 mL). The aqueous layer was extracted further with CH₂Cl₂ (2×50 mL). The combined organics were dried over MgSO₄, filtered and concentrated yielding an off white solid (5.1 g, 93%). LCMS (m/z): 188.0/190.0 (MH⁺).

### Synthesis of methyl[5-(4,4,5,5-tetramethyl(1,3,2-dioxaborolan-2-yl))pyrimidin-2-yl]amine

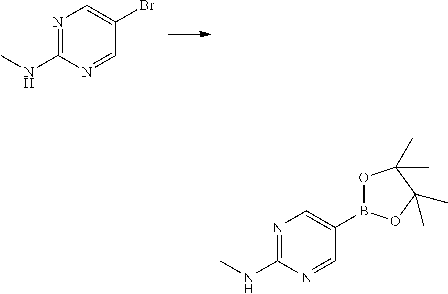

To a dry 500 mL flask was added 2-methylamino-5-bromopyrimidine (9.5 g, 50.5 mmol), potassium acetate (15.1 g, 154.4 mmol), 4,4,5,5,-tetramethyl-2-(4,4,5,5,-tetramethyl-1,3,2-dioxaborolan-2-yl)-1,3,2-dioxaborolane (14.1 g, 55.5 mmol) and dioxane (280 mL). Argon was bubbled through the solution for 15 minutes, at which time 1,1′-bis(diphenylphosphino)ferrocene palladium(II) chloride dichloromethane adduct (2.05 g, 2.51 mmol) was added. The reaction was refluxed in a 115° C. oil bath for 4 hours under argon. After cooling to room temperature, EtOAc (500 mL) was added and the resulting slurry was sonicated and filtered. Additional EtOAc (500 mL) was used to wash the solid. The combined organics were washed with H₂O (2×300 mL), NaCl_{(sat.)}, (300 mL), dried over Na₂SO₄, filtered and the solvents were removed in vacuo. Purification by SiO₂ chromatography (50% EtOAc/hexanes) yielded an off white solid (7.66 g, 64%).

LCMS (m/z): 154 (MH⁺ of boronic acid, deriving from in situ product hydrolysis on LC). ¹H NMR (CDCl₃): δ 8.58 (s, 2H), 5.56 (s, 1H), 3.02 (d, 3H), 1.32 (s, 12H).

### Method 3

### Synthesis of 5-bromo-4-(trifluoromethyl)-2-pyridylamine

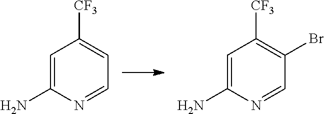 [SMILES: Nc1cc(C(F)(F)F)ccn1 ; Nc1cc(C(F)(F)F)c(Br)cn1]

To a solution of 2-amino-4-trifluoromethylpyridine (10.0 g, 62.1 mmol) in chloroform (200 mL) was added N-bromosuccinimide (12.0 g, 67.4 mmol). The solution was stirred in the dark for 2 hours, at which time it was added to CH₂Cl₂ (200 mL) and 1N NaOH (200 mL). Upon mixing, the layers were separated and the organic layer was washed with NaCl_{(sat.)} (100 mL), dried over Na₂SO₄, filtered and concentrated. The crude material was purified by SiO₂ chromatography (0-5% EtOAc/CH₂Cl₂) yielding 12.0 g (80%) of 5-bromo-4-(trifluoromethyl)-2-pyridylamine LCMS (m/z): 241/243 (MH⁺). ¹H NMR (CDCl₃): δ 8.28 (s, 1H), 6.77 (s, 1H), 4.78 (bs, 2H).

### Synthesis of 5-(4,4,5,5-tetramethyl(1,3,2-dioxaborolan-2-yl))-4-(trifluoromethyl)-2-pyridylamine

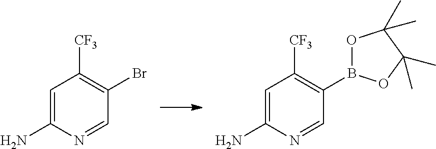 [SMILES: Nc1cc(C(F)(F)F)c(Br)cn1 ; CC1(C)OB(c2cnc(N)cc2C(F)(F)F)OC1(C)C]

To a dry 500 mL flask was added 5-bromo-4-(trifluoromethyl)-2-pyridylamine (11.8 g, 49.0 mmol), potassium acetate (14.4 g, 146.9 mmol), 4,4,5,5-tetramethyl-2-(4,4,5,5-tetramethyl-1,3,2-dioxaborolan-2-yl)-1,3,2-dioxaborolane (13.6 g, 53.9 mmol) and dioxane (300 mL). Argon was bubbled through the solution for 15 minutes, at which time 1,1′-bis(diphenylphosphino)ferrocene palladium(II) chloride dichloromethane adduct (2.0 g, 2.45 mmol) was added. The reaction was refluxed in a 115° C. oil bath for 8 hours under argon. After cooling to room temperature, the dioxane was removed in vacuo. EtOAc (500 mL) was added, and the resulting slurry was sonicated and filtered. Additional EtOAc (500 mL) was used to wash the solid. The combined organic extracts were concentrated and the crude material was partially purified by SiO₂, chromatography (30-40% EtOAc/Hexanes). Upon removal of solvent, hexanes (75 mL) was added; after sonication, the resulting solid was filtered and dried on a high vacuum for 3 days yielding 2.4 g of an off-white solid. By ¹H NMR the material was a 5:1 mixture of boronate ester and 2-amino-4-trifluoromethyl pyridine byproduct. The material was used as is in subsequent Suzuki reactions.

LCMS (m/z): 207 (MH⁺ of boronic acid, deriving from in situ product hydrolysis on LC). ¹H NMR (CDCl₃): δ 8.50 (s, 1H), 6.72 (s, 1H), 4.80 (bs, 2H), 1.34 (s, 12H).

### Method 4

### Synthesis of 5-bromo-4-(trifluoromethyl)pyrimidin-2-amine

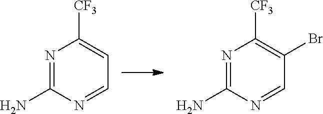 [SMILES: Nc1nccc(C(F)(F)F)n1 ; Nc1ncc(Br)c(C(F)(F)F)n1]

To a solution of 2-amino-4-trifluoromethylpyrimidine (8.0 g, 49.1 mmol) in chloroform (300 mL) was added N-bromosuccinimide (8.9 g, 50 mmol). The solution was stirred in the dark for 16 hours, at which time additional N-bromosuccinimide (4.0 g, 22.5 mmol) was added. After stirring for an additional 4 hours the solution was added to CH₂Cl₂ (200 mL) and 1N NaOH (200 mL). Upon mixing, the layers were separated and the organic layer was washed with NaCl_{(sat.)} (100 mL), dried over Na₂SO₄, filtered and concentrated, yielding 10.9 g (82%) of 5-bromo-4-(trifluoromethyl)-2-pyrimidylamine. LCMS (m/z): 242/244 (MH⁺). ¹H NMR (CDCl₃): δ 8.52 (s, 1H), 5.38 (bs, 2H).

### Synthesis of 5-(4,4,5,5-tetramethyl(1,3,2-dioxaborolan-2-yl))-4-(trifluoromethyl)pyrimidine-2-ylamine

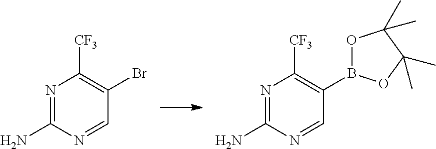 [SMILES: Nc1ncc(Br)c(C(F)(F)F)n1 ; CC1(C)OB(c2cnc(N)nc2C(F)(F)F)OC1(C)C]

To a dry 500 mL flask was added 5-bromo-4-(trifluoromethyl)-2-pyrimidylamine (10.1 g, 41.7 mmol), potassium acetate (12.3 g, 125.2 mmol), 4,4,5,5-tetramethyl-2-(4,4,5,5-tetramethyl-1,3,2-dioxaborolan-2-yl)-1,3,2-dioxaborolane (11.6 g, 45.9 mmol) and dioxane (150 mL). Argon was bubbled through the solution for 15 minutes, at which time 1,1′-bis(diphenylphosphino)ferrocene palladium (II) chloride (1.7 g, 2.1 mmol) was added. The reaction was refluxed in a 115° C. oil bath for 6 hours under argon. After cooling to room temperature, the dioxane was removed in vacuo. EtOAc (500 mL) was added and the resulting slurry was sonicated and filtered. Additional EtOAc (500 mL) was used to wash the solid. The combined organic extracts were concentrated and the crude material was purified by SiO₂, chromatography (30-40% EtOAc/hexanes) yielding 4.40 g of an off white solid. By ¹H NMR the material was a 1:1 mixture of boronate ester and 2-amino-4-trifluoromethylpyrimidine byproduct. The material was used as is in subsequent Suzuki reactions. LCMS (m/z): 208 (MH⁺ of boronic acid, deriving from in situ product hydrolysis on LC). ¹H NMR (CDCl₃): δ 8.72 (s, 1H), 5.50 (bs, 2H), 1.34 (s, 12H).

### Method 5

### Synthesis of 5-bromo-4-chloro-2-pyridylamine

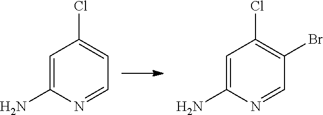 [SMILES: Nc1cc(Cl)ccn1 ; Nc1cc(Cl)c(Br)cn1]

To a solution of 4-chloro-2-pyridylamine (6.0 g, 46.7 mmol) in chloroform (180 mL) was added N-bromosuccinimide (8.3 g, 46.7 mmol). The solution was stirred in the dark for 2 hours, at which time it was added to CH₂Cl₂ (800 mL) and 1N NaOH (100 mL). Upon mixing, the layers were separated and the organic layer was washed with NaCl_{(sat.)} (100 mL), dried over Na₂SO₄, filtered and concentrated. The crude material was purified by SiO₂ chromatography (25-35% EtOAc/hexanes) yielding 3.63 g (38%) of 5-bromo-4-chloro-2-pyridylamine. LCMS (m/z): 206.9/208.9 (MH⁺). ¹H NMR (CDCl₃): δ 8.18 (s, 1H), 6.62 (s, 1H), 4.52 (bs, 2H).

### Synthesis of 4-chloro-5-(4,4,5,5-tetramethyl(1,3,2-dioxaborolan-2-yl))-2-pyridylamine

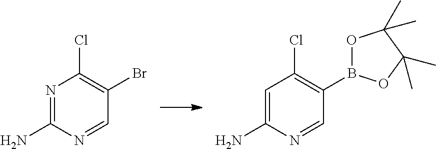 [SMILES: Nc1ncc(Br)c(Cl)n1 ; CC1(C)OB(c2cnc(N)cc2Cl)OC1(C)C]

To a dry 500-mL flask was added 5-bromo-4-chloro 2-pyridylamine (7.3 g, 35.8 mmol), potassium acetate (10.3 g, 105 mmol), 4,4,5,5-tetramethyl-2-(4,4,5,5-tetramethyl-1,3,2-dioxaborolan-2-yl)-1,3,2-dioxaborolane (10.1 g, 39.8 mmol) and dioxane (150 mL). Argon was bubbled through the solution for 15 minutes, at which time 1,1′-bis(diphenylphosphino)ferrocene palladium(II) chloride dichloromethane adduct (0.85 g, 1.04 mmol) was added. The reaction was refluxed in a 115° C. oil bath for 6 hours under argon. After cooling to room temperature, the dioxane was removed in vacuo. EtOAc (500 mL) was then added and the resulting slurry was sonicated and filtered. Additional EtOAc (500 mL) was used to wash the solid. The combined organic extracts were concentrated and the crude material was purified by SiO₂ chromatography (EtOAc as eluent). Upon removal of solvent, 3:1 hexanes/CH₂Cl₂ was added (100 mL). After sonication, the resulting solid was filtered and concentrated in vacuo yielding 2.8 g of a white solid. By ¹H NMR the material was a 10:1 mixture of boronate ester and 2-amino-4-chloropyridine byproduct. The material was used as is in subsequent Suzuki reactions. LCMS (m/z): 173 (MH⁺ of boronic acid, deriving from in situ product hydrolysis on LC). ¹H NMR (CDCl₃): δ 8.36 (s, 1H), 6.46 (s, 1H), 4.70 (bs, 2H), 1.38 (s, 12H).

### Method 6

### Synthesis of 5-bromopyrimidine-2,4-diamine

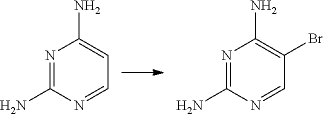 [SMILES: Nc1ccnc(N)n1 ; Nc1ncc(Br)c(N)n1]

To a solution of 2,4-diaminopyrimidine (1.0 g, 9.1 mmol) in chloroform (30 mL) was added N-bromosuccinimide (1.62 g, 9.08 mmol). The solution was stirred in the dark for 12 hours, at which time it was added to CH₂Cl₂ (150 mL) and 1N NaOH (50 mL). The solid that formed was filtered, rinsed with water and concentrated in vacuo, yielding 1.4 g (74%) of 5-bromopyrimidine-2,4-diamine. LCMS (m/z): 189/191 (MH⁺). ¹H NMR (DMSO-_{d6}): δ 7.78 (s, 1H), 6.58 (bs, 2H), 6.08 (bs, 2H).

### Synthesis of 5-(4,4,5,5-tetramethyl-1,3,2-dioxaborolan-2-yl)pyrimidine-2,4-diamine

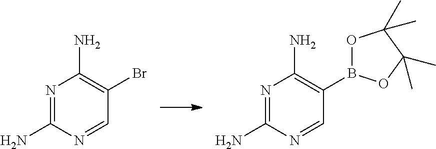 [SMILES: Nc1ncc(Br)c(N)n1 ; CC1(C)OB(c2cnc(N)nc2N)OC1(C)C]

To a dry 1 L flask was added 5-bromopyrimidine-2,4-diamine (30.0 g, 158.7 mmol), potassium acetate (45.8 g, 466.7 mmol), 4,4,5,5-tetramethyl-2-(4,4,5,5-tetramethyl-1,3,2-dioxaborolan-2-yl)-1,3,2-dioxaborolane (51.2 g, 202.2 mmol) and dioxane (500 mL). Argon was bubbled through the solution for 15 minutes, at which time 1,1′-bis(diphenylphosphino)ferrocene palladium(II) chloride (2.5 g, 3.11 mmol) was added. The reaction was refluxed in a 115° C. oil bath for 16 hours under argon. After cooling to room temperature, the solid inorganic material was filtered, rinsed with EtOAc (1 L). The organic filtrate was concentrated in vacuo and to the resulting solid was added dichloromethane (1 L). After sonication the solid was filtered. The solid was the debrominated 2,4-diaminopyrimidine. The filtrate containing desired boronate ester was concentrated in vacuo. To this residue was added diethyl ether (100 mL). After sonication, the solution was filtered, rinsed with additional diethyl ether (50 mL) and the solid obtained was dried under high vacuum to yield the desired 2,4-diaminopyrimidyl-5-boronate ester (10.13 g, 27%). By ¹H NMR the material was a 4:1 mixture of 2,4-diaminopyrimidyl-5-boronate ester and 2,4-diaminopyrimidine byproduct. The material was used as is in subsequent Suzuki reactions. LCMS (m/z): 155 (MH⁺ of boronic acid, deriving from in situ product hydrolysis on LC). ¹H NMR (CDCl₃+CD₃OD): δ 8.16 (s, 1H), 1.34 (s, 12H).

### Method 7

### Synthesis of 5-bromo-6-fluoro-2-pyridylamine

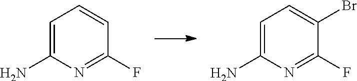 [SMILES: Nc1cccc(F)n1 ; Nc1ccc(Br)c(F)n1]

To a solution of 6-fluoro-2-pyridylamine (1.0 g, 8.93 mmol) in chloroform (55 mL) was added N-bromosuccinimide (1.59 g, 8.93 mmol). The solution was stirred in the dark for 15 hours, at which time it was added to CH₂Cl₂ (200 mL) and 1N NaOH (50 mL). Upon mixing, the layers were separated and the organic layer was washed with NaCl_{(sat.)} (50 mL), dried over Na₂SO₄, filtered and concentrated. The crude material was purified by SiO₂ chromatography (25% EtOAc/hexanes) yielding 5-bromo-6-fluoro-2-pyridylamine (386 mg, 22%). LCMS (m/z): 190.9/192.9 (MH⁺); ¹H NMR (CDCl₃): δ 7.59 (t, J=8.7 Hz, 1H), 6.25 (dd, J=8.1, 1.2 Hz, 1H), 4.58 (bs, 1H).

### Synthesis of 6-fluoro-5-(4,4,5,5-tetramethyl(1,3,2-dioxaborolan-2-yl))-2-pyridylamine

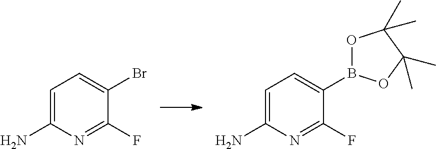 [SMILES: Nc1ccc(Br)c(F)n1 ; CC1(C)OB(c2ccc(N)nc2F)OC1(C)C]

To a dry 50-mL flask was added 5-bromo-6-fluoro-2-pyridylamine (370 mg, 1.93 mmol), potassium acetate (569 mg, 5.8 mmol), 4,4,5,5-tetramethyl-2-(4,4,5,5-tetramethyl-1,3,2-dioxaborolan-2-yl)-1,3,2-dioxaborolane (538 mg, 2.12 mmol) and dioxane (15 mL). Argon was bubbled through the solution for 15 minutes, at which time 1,1′-bis(diphenylphosphino)ferrocene palladium(II) chloride dichloromethane adduct (79 mg, 0.09 mmol). The reaction was refluxed in a 115° C. oil bath for 4 hours under argon. After removal of the volatiles in vacuo, EtOAc (150 mL) was added and the solution was washed with H₂O (3×40 mL), with NaCl_{(sat.)} (300 mL), dried over Na₂SO₄, filtered and concentrated. Purification by SiO₂ chromatography (30% EtOAc/hexanes) yielded boronate ester (161 mg, 35%). LCMS (m/z): 157 (MH⁺ of boronic acid, deriving from in situ product hydrolysis on LC) ¹H NMR (CDCl₃): δ 7.86 (t, J=8.4 Hz, 1H), 6.29 (dd, J=8.1, 2.7 Hz, 1H), 4.70 (bs, 1H), 1.32 (s, 12H).

### Method 8

### Synthesis of 5-bromo-4-fluoropyridin-2-amine

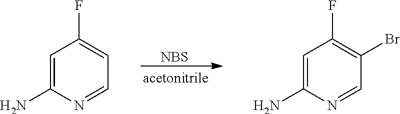

N-Bromosuccinimide (126 mg, 0.71 mmol) was added to a solution of 4-fluoropyridin-2-amine TFA salt (162 mg, 0.72 mmol) in acetonitrile (4 mL) in an aluminum foil-wrapped flask in a darkened hood. The reaction solution was stirred at room temperature in darkness for 2 hours. After evaporation of the solvent, the crude product was purified on a silica gel column eluting with EtOAc to give 5-bromo-4-fluoropyridin-2-amine as an ivory solid (92 mg, 67%). LC/MS (m/z): 190.9/192.9 (MH⁺), Rₜ 1.02 minutes.

### Synthesis of 4-fluoro-5-(4,4,5,5-tetramethyl-1,3,2-dioxaborolan-2-yl)pyridin-2-amine

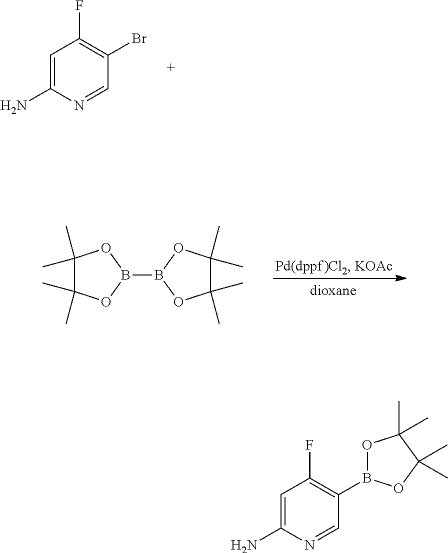

In a sealable Pyrex pressure vessel, a mixture of 5-bromo-4-fluoropyridin-2-amine (25 mg, 0.13 mmol), 4,4,5,5-tetramethyl-2-(4,4,5,5-tetramethyl-1,3,2-dioxaborolan-2-yl)-1,3,2-dioxaborolane (40 mg, 0.16 mmol), potassium acetate (51 mg, 0.52 mmol) and dichloro[1,1′-bis(diphenylphosphino)ferrocene]palladium(II)-dichloromethane adduct (16 mg, 0.019 mmol) was suspended in dioxane (1.7 mL) under argon. The pressure vessel was sealed and the reaction mixture was stirred at 110° C. for 2 hours. After the reaction was complete as judged by LCMS, the reaction mixture was cooled to room temperature and the 4-fluoro-5-(4,4,5,5-tetramethyl-1,3,2-dioxaborolan-2-yl)pyridin-2-amine was used in subsequent reactions without further purification, assuming a quantitative yield (0.13 mmol). LC/MS (m/z): 157.0 (MH⁺ of the boronic acid formed by product hydrolysis on LC), Rₜ 0.34 minutes.

### Method 9

### Synthesis of 2-amino-5-bromo-isonicotinonitrile

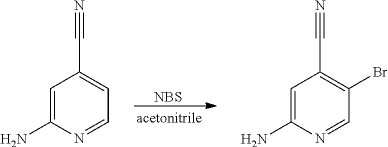

In an aluminum foil-covered flask in a darkened hood, 2-amino-isonicotinonitrile TFA salt (125 mg, 0.54 mmol) was dissolved in acetonitrile (3.5 mL). Solid N-bromosuccinimide (89.2 mg, 0.501 mmol) was added to the stirred solution in one portion at RT. The reaction solution was stirred at room temperature in darkness for 90 minutes. After evaporation of the solvent, the crude material was further purified by silica gel chromatography to give 2-amino-5-bromo-isonicotinonitrile (53 mg, 49%). LC/MS (m/z): 197.9 (MH⁺), Rₜ 2.92 minutes.

### Synthesis of 2-amino-5-boronic ester-isonicotinonitrile

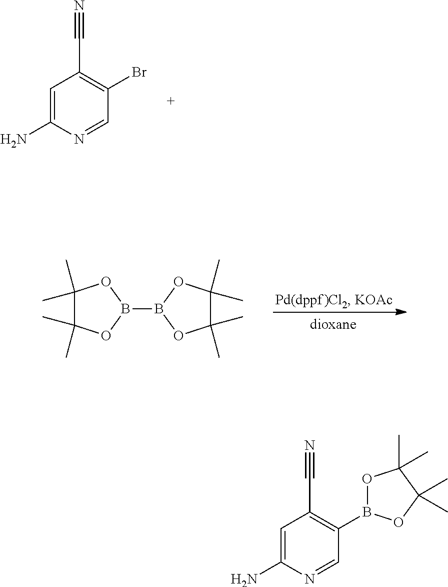

In a glass pressure vessel, a mixture of 2-amino-5-bromo-isonicotinonitrile (25 mg, 0.126 mmol), 4,4,5,5-tetramethyl-2-(4,4,5,5-tetramethyl-1,3,2-dioxaborolan-2-yl)-1,3,2-dioxaborolane (38 mg, 0.151 mmol) and potassium acetate (49 mg, 0.504 mmol) were suspended in dioxane (1.8 mL). After purging the mixture with argon for 1-2 min, dichloro[1,1′-bis(diphenylphosphino)ferrocene]palladium(II) dichloromethane adduct (16 mg, 0.019 mmol) was added in one portion. The reaction vessel was sealed and heated at 120° C. with stirring for 2 hours. The crude reaction solution was cooled to room temperature and used without further purification assuming a quantitative yield of the boronic ester (0.126 mmol). LC/MS (m/z): 164.0 (MH⁺ of the boronic acid formed by product hydrolysis on LC), Rₜ 0.37 minutes.

### Method 10

### Synthesis of 5-fluoro-2-morpholinopyrimidine-4,6-diol

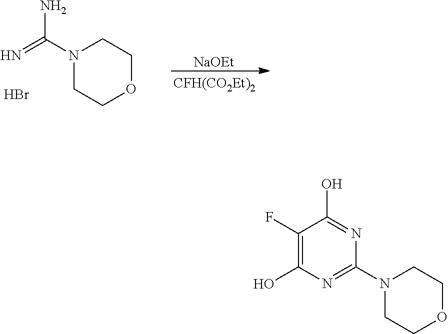 [SMILES: Br ; N=C(N)N1CCOCC1 ; Oc1nc(N2CCOCC2)nc(O)c1F ; "NaOEt"]

Sodium hydride (60% in oil, 3.9 g, 96.5 mmol) was washed with hexanes in a round bottom flask under argon and cooled with an ice water bath. EtOH (100 mL) was slowly added. The resulting mixture was warmed to RT and stirred for 30 minutes. To the base mixture, diethyl 2-fluoromalonate (5.7 g, 32.2 mmol) was added, followed by morpholinoformamidine hydrobromide (6.8 g, 32.2 mmol). The mixture was heated to 90-95° C. with stirring under argon. After 12 hours, the reaction was cooled to room temperature and the EtOH was removed in vacuo. The resulting white solid was dissolved in water (25 mL) and acidified with conc. HCl to pH=3-4. A white precipitate formed which was collected on a Büchner filter, washed with water (2×50 mL), air dried on the filter, and dried in vacuo to give 5-fluoro-2-morpholinopyrimidine-4,6-diol (0.87 g, 12%).

LC/MS (m/z): 216.0 (MH⁺), Rₜ 0.63 minutes.

### Synthesis of 4-(4,6-dichloro-5-fluoropyrimidin-2-yl)morpholine

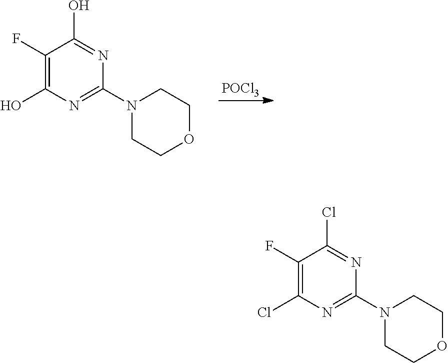 [SMILES: Oc1nc(N2CCOCC2)nc(O)c1F ; "POCl3" ; Fc1c(Cl)nc(N2CCOCC2)nc1Cl]

A mixture of 5-fluoro-2-morpholinopyrimidine-4,6-diol (0.87 g, 4.0 mmol) and POCl₃ (10 mL) was heated at 120° C. for 16 hours, then cooled to RT. Excess of POCl₃ was removed under reduced pressure to give a semi-solid which was dried further in vacuo. After 12 h of vacuum drying, the solid was diluted in EtOAc (150 mL) and washed with sat. NaHCO₃ (60 mL). A solid formed during the wash and was discarded with the aqueous layer. The organic layer was washed again with sat. NaHCO₃ (2×30 mL), brine (30 mL), dried with Na₂SO₄, filtered and evaporated under reduced pressure to give a crude product. The product was purified by flash chromatography eluting with 25% EtOAc/hexane to give 4-(4,6-dichloro-5-fluoropyrimidin-2-yl)morpholine (418 mg, 42%). LC/MS (m/z): 251.9 (MH⁺), Rₜ 3.22 minutes.

### Method 11

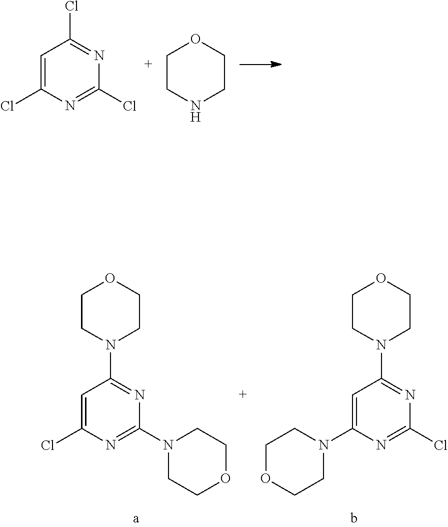

A solution of morpholine (100 g; 1.15 moles; 5.3 equivalents) in THF (450 mL) was cooled with an ice bath. A solution of 2,4,6-trichloropyrimidine (39.9 g; 217 mmoles; 1.0 equivalents) in THF (100 mL) was added over a period of 30 minutes. A copious white precipitate formed upon addition of 2,4,6-trichloropyrimidine and the reaction mixture rapidly thickened. The mixture was allowed to warm to ambient temperature and mechanically stirred for 64 hours (heating the reaction mixture at reflux following the addition of 2,4,6-trichloropyrimidine leads to complete reaction in 60 min. The ratio of a to b was unchanged). The mixture was then filtered and the filter cake washed with additional THF (2×100 mL). The filtrate was concentrated on the rotavap. Water (600 mL) was added and the resulting slurry was stirred for 30 minutes. The solids were isolated by filtration, washed with additional water (2×100 mL) and dried overnight under vacuum. Yield a+b: 61.3 g (99%). Product was 87% a by hplc area percent; remainder is b.

31 g of the crude solid was dissolved in 200 mL of CH₂Cl₂ and applied to 600 g of dry silica in a fritted glass funnel. The silica was eluted with 1:1 hexane:EtOAc and 300 mL fractions were collected. TLC analysis shows a to be present in fractions 1-7 and 4,6-dimorpholino-2-chloropyrimidine in fractions 6-10. Fractions 1-5 were pooled and concentrated to provide a white solid. Yield: 28.2 g (Product was 98% a by hplc area percent).

### Example 1

### Preparation of 4-(Trifluoromethyl)-5-(2,6-dimorpholinopyrimidin-4-yl)pyridin-2-amine

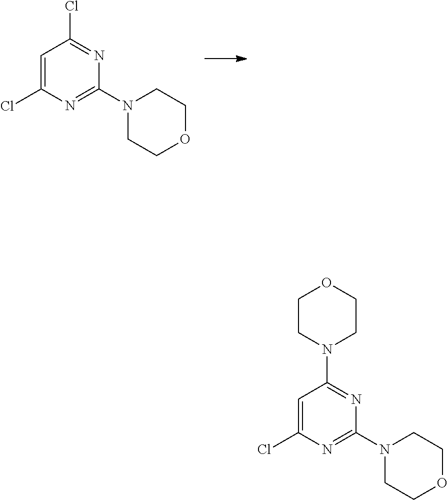 [SMILES: Clc1cc(Cl)nc(N2CCOCC2)n1 ; Clc1cc(N2CCOCC2)nc(N2CCOCC2)n1]

To a slurry of 2-morpholino-4,6-dichloropyrimidine (prepared as in Method 22, 2.0 g, 8.54 mmol) in NMP (14 mL), triethylamine (1.43 mL, 10.25 mmol) was added. The heterogeneous mixture was stirred for 15 minutes, then treated with morpholine (0.75 mL, 8.54 mmol). Upon refluxing at 85° C. under argon for 2 hours, the solution was cooled, then added to EtOAc (160 mL). The organic solution was washed with 25 mL of NaHCO_{3(sat.)} (2×), water (2×) and brine, dried over Na₂SO₄, filtered and concentrated. The crude material was dissolved in 200 mL EtOAc and filtered through a SiO₂ pad, further eluting with EtOAc, yielding 2.2 g (93%) of 2,4-dimorpholino-6-chloropyrimidine as an off-white solid. LCMS (m/z): 285.0 (MH⁺), ¹H NMR (CDCl₃): δ 5.86 (s, 1H), 3.71-3.76 (m, 12H), 3.52-3.56 (m, 4H).

### 4-(Trifluoromethyl)-5-(2,6-dimorpholinopyrimidin-4-yl)pyridin-2-amine

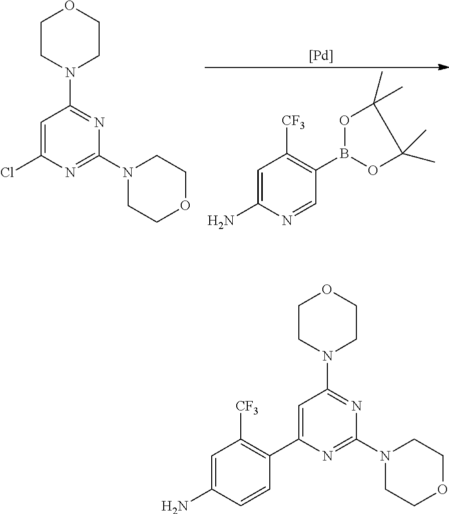

Argon gas was bubbled through a heterogeneous mixture of 2,4-dimorpholino-6-chloropyrimidine (4.1 g, 14.3 mmol) and 4-(trifluoromethyl)-5-(4,4,5,5-tetramethyl-1,3,2-dioxaborolan-2-yl)pyridin-2-amine (16.5 g, 57.3 mmol) in 1,2-dimethoxyethane and 2M Na₂CO₃ (3:1) for 20 minutes. 1,1′-Bis(diphenylphosphino)ferrocene palladium (II) chloride (292 mg, 0.36 mmol) was added and the high pressure glass vessel containing the mixture was sealed. The reaction mixture was then heated at 90° C. for 15 hours, cooled and diluted with EtOAc (300 mL). The organic solution was washed with 300 mL of a mixture of water: Na₂CO_{3(sat.)}:NH₄OH_{(conc.)}=5:4:1, then NH₄Cl_{(sat.)}, and brine (2×), dried over Na₂SO₄, filtered and concentrated. The crude material was purified by SiO₂ chromatography (50-90% EtOAc/hexanes with 0.1% TEA) resulting in 5.62 g (95%) of 4-(trifluoromethyl)-5-(2,6-dimorpholinopyrimidin-4-yl)pyridin-2-amine as an off-white solid. LCMS (m/z): 411.3 (MH⁺); ¹H NMR (CDCl₃): δ 8.27 (s, 1H), 6.78 (s, 1H), 5.97 (s, 1H), 4.77 (bs, 2H), 3.59-3.80 (m, 12H), 3.58-3.61 (m, 4H).

### Example 2

### Test formulation for 4-(trifluoromethyl)-5-(2,6-dimorpholinopyrimidin-4-yl)pyridin-2-amine

4-(Trifluoromethyl)-5-(2,6-dimorpholinopyrimidin-4-yl)pyridin-2-amine powder was dissolved in 1 volume of NMP (1-methyl-2-pyrrolidone). After dissolution (if needed, in warm water), add 9 volumes of PEG300. The final ratio is: NMP 10%/PEG300 90%.

### Example 3

### The Role of p110a and/or p110b in the Development of Pten Hamartoma Tumor Syndrome or PHTS

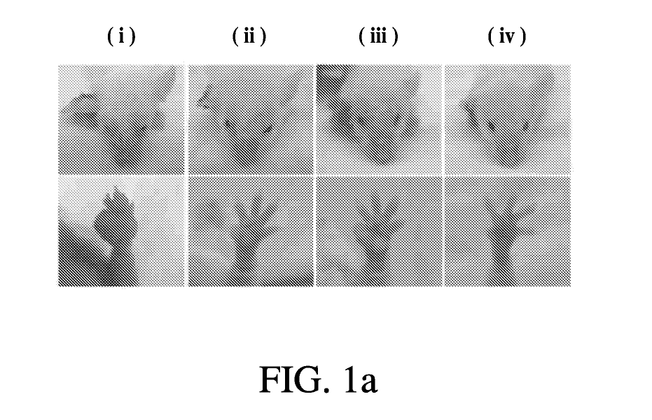

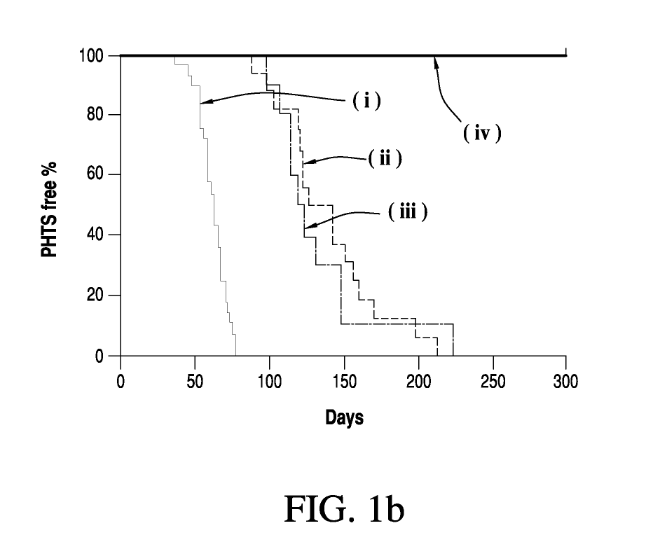

Pten f/f mice (Lesche, R., et al., Cre/loxP-mediated inactivation of the murine Pten tumor suppressor gene. Genesis, 2002. 32(2): 148-9) were crossed with K14-cre mice (Jonkers, J., et al., Synergistic tumor suppressor activity of BRCA2 and p53 in a conditional mouse model for breast cancer. Nat Genet, 2001. 29(4): 418-25) to generate K14-Cre Pten f/f mice in which the floxed Pten allele is deleted specifically in the keratinocytes by the K14-driven Cre recombinase. These mice were further crossed with p110a f/f (Zhao, J. J., et al., The p110 alpha isoform of PI3K is essential for proper growth factor signaling and oncogenic transformation. Proc Natl Acad Sci USA, 2006. 103(44): 16296-300) and p110b f/f mice (Jia, S., et al., Essential roles of PI(3)K-p110beta in cell growth, metabolism and tumorigenesis. Nature, 2008) to generate K14-cre Pten f/f, K14-cre Pten f/f; p110a f/f, K14-cre Pten f/f; p110b f/f, and K14-cre-Pten f/f; p110a f/f; p110b f/f mice. Keratinocyte-specific deletion of Pten results in multiple dermal lesions in the K14-cre Ptenf/f mice that resemble multiple skin hamartomas of PHTS. Panel (a) in FIG. 1 illustrate the heads and front paws of mice of the relevant genotypes as indicated at 12 weeks of age. All mice are on an FVB background. Additional ablation of either p110a or p110b delayed onset and reduces the severity of the lesions and ablation of both p110 isoforms blocks the appearance of symptoms as demonstrated visually in the photographs in the (a) panel.

FIG. 1, panel (b) is a Kaplan-Meier plot of the onset of PHTS in the K14-cre Pten f/f (n=28), K14-cre Pten f/f; p110a f/f (n=16), K14-cre Pten f/f; p110b f/f (n=11) and k14-cre-Pten f/f; p110a f/f; p110b f/f (n=15) mice. The median PHTS onset for K14-cre Pten f/f mice (red line) is 62 days. Ablation of either p110a (green line) or p110b (blue line) delays symptom onset by about 60 days although all mice of these genotypes display symptoms by about 210-225 days. In striking contrast, all of the K14-cre-Pten f/f; p110a f/f; p110b f/f mice remained free of PHTS symptoms for at least 300 days.

### Example 4

### Early Administration of a Compound of Formula (I) Prevents the Development of PHTS Symptoms in K14-cre-Pten f/f Mice

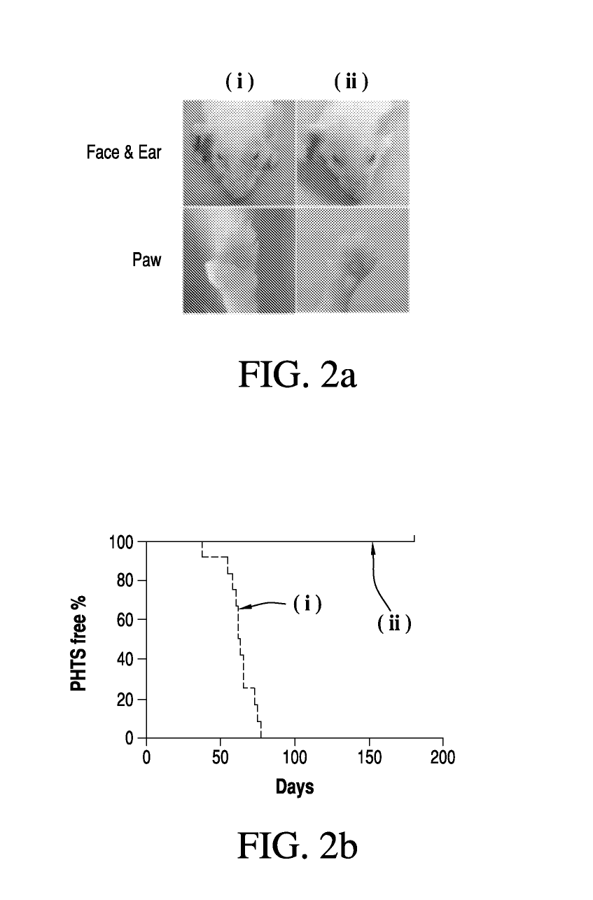

K14-cre-Pten f/f mice were treated daily with 25 mg/kg 4-(trifluoromethyl)-5-(2,6-dimorpholinopyrimidin-4-yl)pyridin-2-amine by oral gavage beginning at 3 weeks of age and the development of PHTS symptoms monitored. FIG. 2, panel (a) demonstrates that mice treated with 4-(trifluoromethyl)-5-(2,6-dimorpholinopyrimidin-4-yl)pyridin-2-amine remained free of PHTS symptoms while mice treated with vehicle alone developed characteristic PHTS lesions on their face and limbs. FIG. 2, panel (b) represents a Kaplan-Meier curve of PHTS free survival in K14-cre-Pten f/f mice (n=12) maintained with 4-(trifluoromethyl)-5-(2,6-dimorpholinopyrimidin-4-yl)pyridin-2-amine as described above or treated with vehicle only.

### Example 5

### Administration of a Compound of Formula (I) Relieves PHTS Symptoms in K14-cre-Pten f/f Mice

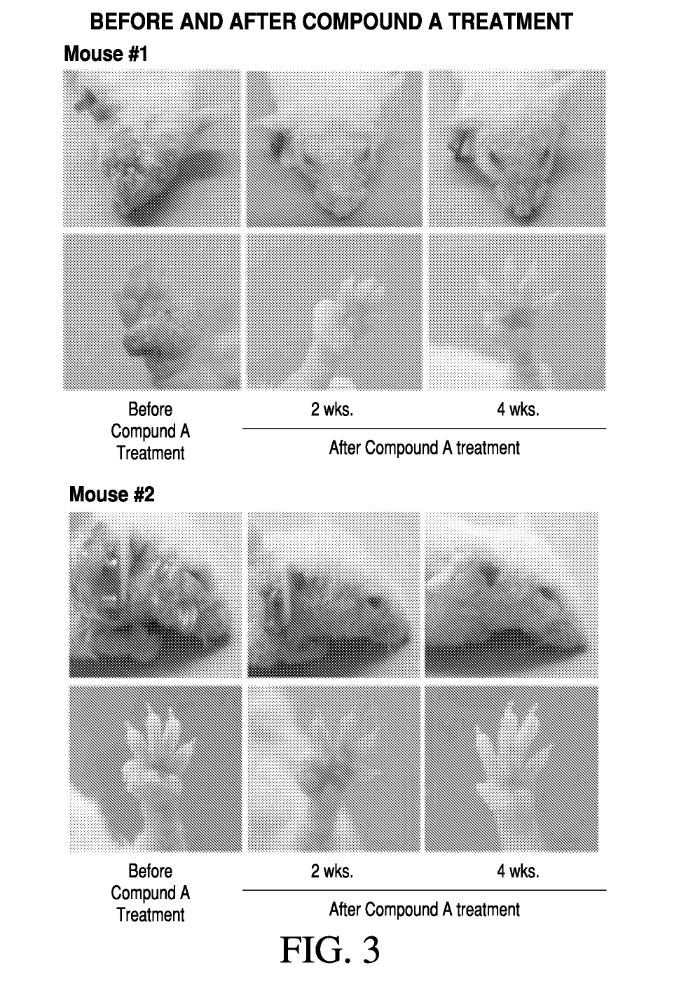

K14-cre-Pten f/f mice that had fully developed PHTS (12-14 weeks of age) were treated daily with 45 mg/kg 4-(trifluoromethyl)-5-(2,6-dimorpholinopyrimidin-4-yl)pyridin-2-amine by oral gavage. The FIG. 3 photographs show the heads and front left paws of two K14-cre-Pten f/f mice treated daily with 45 mg/ml 4-(trifluoromethyl)-5-(2,6-dimorpholinopyrimidin-4-yl)pyridin-2-amine as described in the legend to FIG. 3. 4-(trifluoromethyl)-5-(2,6-dimorpholinopyrimidin-4-yl)pyridin-2-amine was administered for 4 weeks and mice were photographed at before treatment, 2 and 4 weeks. Administration of 4-(trifluoromethyl)-5-(2,6-dimorpholinopyrimidin-4-yl)pyridin-2-amine dramatically relieved the PHTS symptoms in these mice. Most PHTS symptoms were substantially or completely reduced at the end of the 4-week 4-(trifluoromethyl)-5-(2,6-dimorpholinopyrimidin-4-yl)pyridin-2-amine treatment.

Results

The findings show that, in an animal model of PHTS, while loss of either p110α or p110β isoform of PI3K significantly reduced the occurrence and severity of PHTS, ablation of both isoforms completely prevented the development of PHTS. The findings further demonstrate that administration of 4-(trifluoromethyl)-5-(2,6-dimorpholinopyrimidin-4-yl)pyridin-2-amine in young mice also entirely blocked appearance of PHTS. More strikingly, administration of 4-(trifluoromethyl)-5-(2,6-dimorpholinopyrimidin-4-yl)pyridin-2-amine in mice with advanced skin lesions completely reversed the phenotype of PHST.

1. A method for inhibiting growth or proliferation of hamartoma tumor cells comprising administering to a patient in need thereof in an amount that is effective to inhibit growth or proliferation of the hamartoma tumor cells a compound of the formula 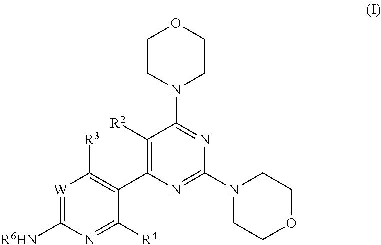
wherein R² is hydrogen or halogen; R³ is hydrogen, cyano, nitro, halogen, hydroxyl, amino, or trifluoromethyl; R⁴ is hydrogen or halogen; R⁶ is hydrogen, methyl, or ethyl; and W is CR_{w} or N, wherein R_{w} is hydrogen, cyano, halogen, methyl, trifluoromethyl, or sulfonamido; or a pharmaceutically acceptable salt thereof.

2. A method according to claim 1 wherein W is CH.

3. A method according to claim 1 wherein R² is hydrogen; R³ is hydrogen or trifluoromethyl; R⁴ is hydrogen; and R⁶ is hydrogen.

4. A method according to claim 3 wherein W is CH.

5. A method according to claim 4 wherein R³ is trifluoromethyl.

6. A method according to claim 1 wherein the compound of formula (I) is 4-(trifluoromethyl)-5-(2,6-dimorpholinopyrimidin-4-yl)pyridin-2-amine or a pharmaceutically acceptable salt thereof.

7. A method according to claim 1 wherein the amount of the compound or salt of formula (I) that is effective to inhibit growth or proliferation of the hamartoma tumor cells is an administered amount ranging from 0.001 to 1000 mg/kg.

8. A method according to claim 6 wherein the amount of the compound or salt of formula (I) that is effective to inhibit growth or proliferation of the hamartoma tumor cells is an administered amount ranging from 1.0 to 1000 mg/kg.

9. A method for treating a PTEN hamartoma tumor syndrome comprising administering to a patient in need thereof in an amount that is effective to inhibit growth or proliferation of the hamartoma tumor cells a compound of the formula 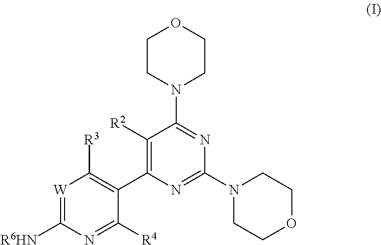
wherein R² is hydrogen or halogen; R³ is hydrogen, cyano, nitro, halogen, hydroxyl, amino, or trifluoromethyl; R⁴ is hydrogen or halogen; R⁶ is hydrogen, methyl, or ethyl; and W is CR_{w} or N, wherein R_{w} is hydrogen, cyano, halogen, methyl, trifluoromethyl, or sulfonamido; or a pharmaceutically acceptable salt thereof.

10. A method according to claim 9 wherein W is CH.

11. A method according to claim 10 wherein R² is hydrogen; R³ is hydrogen or trifluoromethyl; R⁴ is hydrogen; and R⁶ is hydrogen.

12. A method according to claim 11 wherein W is CH.

13. A method according to claim 12 wherein R³ is trifluoromethyl.

14. A method according to claim 9 wherein the compound of formula (I) is 4-(trifluoromethyl)-5-(2,6-dimorpholinopyrimidin-4-yl)pyridin-2-amine or a pharmaceutically acceptable salt thereof.

15. A method according to claim 9 wherein the amount of the compound or salt of formula (I) that is effective to inhibit growth or proliferation of the hamartoma tumor cells is an administered amount ranging from 0.001 to 1000 mg/kg.

16. A method according to claim 14 wherein the amount of the compound or salt of formula (I) that is effective to inhibit growth or proliferation of the hamartoma tumor cells is an administered amount ranging from 1.0 to 30 mg/kg.

17. A method according to claim 9 wherein the PTEN hamartoma tumor syndrome is selected from the group consisting of Cowden syndrome, Lhemitte-Duclos disease, Bannayan-Riley-Ruvalcaba syndrome, and Proteus syndrome.

18. A method according to claim 9 wherein the PTEN hamartoma tumor syndrome is Cowden syndrome.

19. A method according to claim 14 wherein the PTEN hamartoma tumor syndrome is selected from the group consisting of Cowden syndrome, Lhemitte-Duclos disease, Bannayan-Riley-Ruvalcaba syndrome, and Proteus syndrome.

20. A method according to claim 14 wherein the PTEN hamartoma tumor syndrome is Cowden syndrome.

21-38. (canceled)
